(19) United States
(12) Patent Application Publication
(10) Pub. No.: US 2021/0030271 A1
(43) Pub. Date: Feb. 4, 2021

(54) Ophthalmic Gonioscopic Lens Device with Tilted Lenses

(71) Applicant: Arkadiy Farberov, Novato, CA (US)

(72) Inventor: Arkadiy Farberov, Novato, CA (US)

(21) Appl. No.: 16/530,959

(22) Filed: Aug. 2, 2019

(51) Int. Cl. A61B 3/117

(52) U.S. Cl. CPC A61B 3/117; A61F 9/008

(57) ABSTRACT

The device of the invention that consists of a gonioscopic lens and gonioscopic lens support for supporting the lens during an ophthalmic procedure. The lens is a cylindrical body with tilted end faces on both end sides. The lens support is a part, which is placed on the patient's eye, while the other side supports the gonioscopic lens. The eye support can be made in different modifications but in any case has a cavity capable of accommodating the eye cornea. The device has two optical lenses. The first lens is a convex lens formed on or attached to the proximal end face of the gonioscopic lens. The second lens is located at the distal end of the gonioscopic lens and may constitute either a concave lens formed on the gonioscopic lens eye support or a concave curvilinear surface of the cavity on the distal end of the gonioscopic lens.

### FIELD OF THE INVENTION

The present invention relates to optical devices for ophthalmology, in particular to gonioscopes for intraocular observation and laser surgery. More specifically, the invention relates to an ophthalmic gonioscopic lens device with tilted lenses for both intraocular observation and laser surgery.

### DESCRIPTION OF THE PRIOR ART

The techniques used for viewing the inner parts of the eye, such as retina and anterior chamber angle of the eye, for evaluation management, and classification of normal and abnormal structures is known as gonioscopy, and devices used for gonioscopy are known as gonioscopes. Observation of the anterior chamber and especially its angle areas, which are difficult or impossible to see with the use of some conventional optical means, is very important for diagnosis of eye diseases. For example, the classification of glaucoma relies heavily upon knowledge of the anterior segment anatomy, particularly that of the anterior chamber angle.

Heretofore, many gonioscopic devices have been known. The basic gonioscopic instrument used in the art is known as a Goldmann “universal” lens and mirrors or Roussel lens assembly. This gonioscope comprises an optical body with flat tapered sides having an entrance face which is flat or spherical, a spherical exit face which is applied to the cornea of the eye, a reflecting face and a compensating element, for example a piano-cylindrical lens. The Goldmann-type gonioscope is a universal three-reflection lens assembly for biomicroscopic investigation and laser coagulation of the eye bottom and the front camera angle of the eye. With the help of lenses by Goldmann in combination with a binocular microscope of a slit lamp a high quality image, a step-by-step observation of the eye bottom up to a tooth-like line, gonioscopy, detection of minute variation of eye structures under observation and spatial-depth localization of pathologic structures are provided.

Many other modifications of the Goldmann gonioscope are known.

The inventor herein has developed a Goldmann-type gonioscope that is described in U.S. Pat. No. 6,942,343 issued on Sep. 13, 2005. This gonioscope comprises a hollow tapered body with mirror surfaces formed on the inner side of the gonioscope or on the inner surfaces of the inserts, which are placed into the recesses on the inner surface of the gonioscope. Several reflecting surfaces can be arranged at the same or different angles to the longitudinal axis of the gonioscope. In the second embodiment, the gonioscope can be made disposable and molded with reflecting surface applied after molding onto the inner flats by evaporation in vacuum, or by other methods. In a third embodiment, the gonioscope is used in combination with a double-concave contact lens which is applied onto the eye cornea and is attached to or is separate from the gonioscope body and is used as a support for sliding the front end of the gonioscope over the lens surface for orientation thereof at different angles to the optical axis of the eye. In a fourth embodiment, the gonioscope is an assembly of the tapered body with the mirror inserts placed into the recesses on the inner surface of the hollow gonioscope.

U.S. Pat. No. 7,419,262 issued to Paul C Whalen on Sep. 2, 2008 discloses a two-component gonioscopic device consisting of an optical wedge-like prism and an eye support onto which the prism rests during the examination.

In one of the embodiments, the prism has on one of its faces a spherical recess at the distal end of the prism. An eye support, which has a contact lens and which during the ophthalmologic process is placed onto the cornea of the eye, has a rear spherical surface, which is brought in contact with the spherical recess of the prism so that the prism may freely roll over the spherical surface of the support for changing the angle of observation of the peripheral parts of the anterior chambers of the eye. The prism has first and second mirror surfaces, which are positioned to redirect light rays from the periphery of the anterior chamber and reflect them in a direction parallel or nearly parallel to the optical axis of the contact lens surface. A first mirror surface is offset from the anterior surface of the contact lens element in an outward direction from the contact optical axis. A second mirror surface is offset from the contact optical axis, preferably in a direction opposite from said first mirror surface and angled away from the contact optical axis as the second mirror surface extends in an anterior direction. Preferably, the first mirror surface is offset from the anterior surface sufficiently to provide a visual gap between the contact lens and the first mirror surface along a sightline parallel to the contact optical axis. U.S. Pat. No. 4,598,984 issued on Jul. 8, 1986 to Pascal Rol discloses a contact lens arrangement for optically examining an eye and/or treating it by light irradiation, in particular the retina, in the region of or away from the optical axis of the eye. The contact lens arrangement comprises a Goldmann lens having a plane entry surface and a spherical exit surface, applied to the cornea of the eye, and a compensating element, e.g. a prism, having an entry surface. The compensating element is fixed to the entry surface of the Goldmann lens and its function is to produce an astigmatism opposite to that of the eye with an incident light beam, entering through its entry surface.

U.S. Pat. No. 7,766,480 issued on to Raymond D. Graham, et al. on Aug. 3, 2010 discloses a gonioscopic lens that includes a contact lens, element and a geometric reference member. The contact lens element has an optical axis, a concave posterior contact surface to be placed in optical contact with the cornea of a patient's eye and an anterior surface. The geometric reference member, such as a scale, is provided rear the concave posterior contact surface of the contact lens element. The configuration and location of the geometric reference member are chosen to provide an image of the geometric reference member adjacent to an image of the periphery of the anterior chamber of the eye of a patient within a field of view of the contact lens element when the contact lens element, is placed on the eye of a patient. The two images are formed relative to each other such that they are simultaneously in focus to permit visual comparison between the two.

U.S. Pat. No. 9,226,658 issued to Steven John et al on Jan. 5, 2016 discloses a nolithic gonioscopic lens prism supported by an elongated handle to which the prism is attached. The device has a stabilization mechanism for stabilizing the prism relative to a patient's eye while permitting movement of the gonioscopic lens relative to the handle. The gonioscopic lens system can be configured to be positioned adjacent a patient's eye using the handle which can be grasped by a user, such as a clinician.

Thus, roughly, gonioscopes used in ophthalmology can be divided into two groups: 1) gonioscope that, are hollow bodies with full internal reflection from mirrors attached to the inner surface of the hollow bodies, and 2) goniscopes having monolithic prisms with full internal reflection from the walls of the monolithic bodies.

### BRIEF DESCRIPTION OF THE DRAWINGS

FIG. 1A is a side view of a gonioscopic device in accordance with one aspect of the invention.

FIG. 1B is a side view of the gonioscopic device of FIG. 1A in a position here the gonioscopic lens rests on the gonioscopic lens support.

FIG. 2 is a side view of the gonioscopic device of FIG. 1A in a working position during an eye examination procedure.

FIG. 3 is a view on the device of FIG. 2 in the direction of arrow A.

FIG. 4 a view on the device of FIG. 2 in the direction of arrow B.

FIG. 5A is a side view of a gonioscopic device similar to FIG. 1A but in accordance with another aspect of the invention, wherein the second lens is a concave surface on the distal end of the gonioscopic lens.

FIG. 5B is a side view of the gonioscopic device of FIG. 5A in a position where the gonioscopic lens rests on the gonioscopic lens support.

FIG. 5C is a view in the direction of arrow A′ of FIG. 5B.

FIG. 5D is a view in the direction of arrow B′ of FIG. 5B.

FIG. 6A is a side view of the device according to another aspect of the invention, in which the second lens is convex and is formed on the rear side of the gonioscopic lens eye support.

FIG. 6B is a view of the device of FIG. 6A in a position where the gonioscopic lens rests on the gonioscopic lens eye support.

FIG. 7 shows a gonioscopic device, which is the same as one shown in FIG. 6B except that the first lens is made integrally with the gonioscopic lens,

FIG. 8 is side view of the gonioscopic device in a working position during the eye examination procedure, wherein the gonioscopic lens is inserted into a funnel-shaped casing and is supported by an inner circular step formed inside the casing.

FIG. 9 is a transverse sectional view of a gonioscopic lens eye support of FIG. 8.

FIG. 10 is a three-dimensional view of a gonioscopic lens eye support with a handle for manipulating with the device.

### SUMMARY OF THE INVENTION

The invention relates to optical devices for ophthalmology, in particular to gonioscopes for intraocular observation and laser surgery. More specifically, the invention relates to an ophthalmic gonioscopic lens device with tilted lenses for both intraocular observation and laser surgery.

The device of the invention consist essentially of two separate parts, i.e., a gonioscopic lens and a gonioscopic lens support for supporting the gonioscopic lens during the ophthalmic procedure. The gonioscopic lens is a transparent body of rotation, which preferably is a cylindrical body with tilted end faces on both ends. Both end faces are titled at predetermined angles with respect to the optical axis of an eye to be observed. The lens is intended for observation of anterior chamber angle of the eye, which is not accessible to view, by conventional optical lenses. The gonioscopic lens eye support is a part of the device, one side of which is placed on the patient's eye, and another side support the gonioscopic lens. The eye support can be made in different modifications but, in any case, it has a cavity capable of accommodating the eye cornea. The device has two optical lenses. In any case, the first lens is a convex lens formed on or attached to the proximal end face of the gonioscopic lens. The second lens is located at the distal end of the gonioscopic lens and may constitute either a concave lens formed on the gonioscopic lens eye support or a concave curvilinear surface of the cavity on the distal end of the gonioscopic lens.

It should be noted that practically all existing gonio lenses work by reflecting the light reflected from inclined flat mirrors of different designs. Therefore, during a procedure, it is necessary to use a slit lamp and, since a zone of illumination is about 2 square mm, it should be observed via a microscope. Contrary to this, in the case of the present invention, the area of the illuminated zone is large enough and is about 20 square mm. Therefore, a microscope is not needed.

### DETAILED DESCRIPTION OF THE INVENTION

The present invention relates to optical devices for ophthalmology, in particular to gonioscopes for intraocular observation and laser surgery. More specifically, the invention relates to an ophthalmic gonioscopic lens device with tilted lenses for both intraocular observation and laser surgery.

The ophthalmic gonioscopic lens device (hereinafter referred to as “gonioscopic device”) relates to a two-component gonioscopic device, i.e., gonioscopic lens and a contact gonioscopic lens eye support (hereinafter referred to as “a gonioscopic lens eye support”), which, during the eye examination, is placed onto a patient's eye with its front side and supports the gonioscopic lens on its rear side or inside of a frame-like eye support.

The gonioscopic device of the invention will be further described with reference to specific embodiments and modifications, which, however, should not be construed as limiting the scope of the application of the invention and are given only as example.

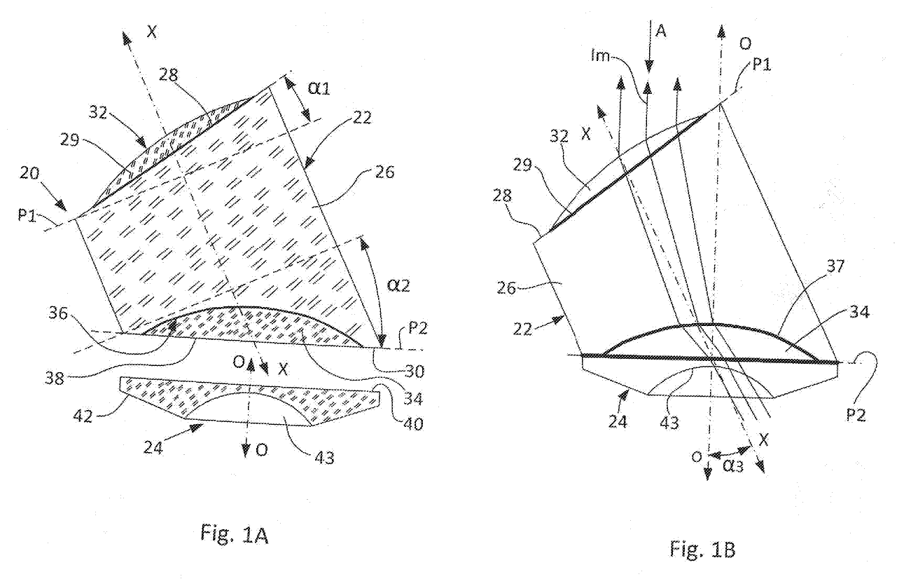

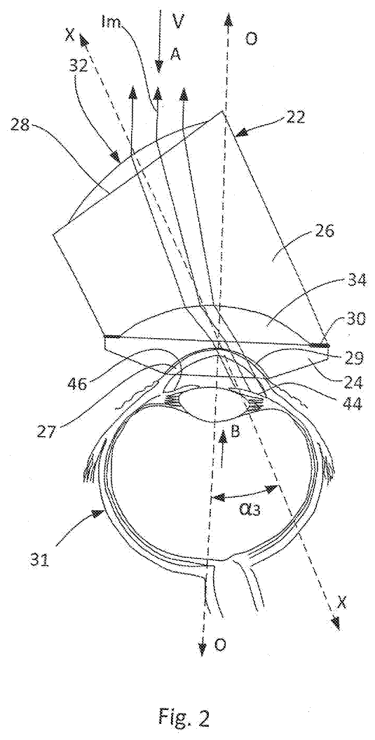

FIG. 1A is a side view of a gonioscopic device in accordance with one aspect of the invention. FIG. 2 is a side view of the gonioscopic device in a working position during the eye examination procedure.

It can be seen from FIG. 1A that the gonioscopic device, which, in general, is designated by reference numeral 20, consists of two parts, i.e., a gonioscopic lens 22 and a gonioscopic lens eye support 24. Although in FIG. 1A both parts are shown separated from each other, during an ophthalmologic procedure both parts are in a position shown in FIG. 1B, where the gonioscopic lens eye support 24 is brought into a gentle contact with the cornea 27 (FIG. 2) of the eye 31 through a thin layer of a cushioning agent 29, and the gonioscopic lens 22 is maintained in optical contact with the rear side of the gonioscopic lens eye support 24. The gonioscopic lens eye support 24 may be disposable or can be made from a sterilizable optical material.

More specifically, the gonioscopic lens is a cylindrical optically transparent body 26. The cylinder has a longitudinal axis X-X (FIGS. 1A and 1B). The cylindrical body 26 has two flat end faces formed by cutting the cylinder ends with planes tilted relative to axis X-X. Thus, on the side of a viewer V who looks in the direction of arrow A (FIG. 2), the gonioscopic lens 22 has a proximal end face 28 tilted at an angle α1 to a plane P1, w (FIG. 1A), which is normal to the axis X-X. On the other hand, on the side of an eye 31, the gonioscopic lens 22 has a distal end face 30 (FIG. 1A) tilted at an angle α2 to a plane P2, which is normal to the axis X-X. Reference numeral O-O designates the central symmetrical axis of the gonioscopic lens eye support 24 and during the procedure coincides with the optical axis of the eye (FIG. 2). Angle α3 shown in FIG. 2 and in FIG. 1B is an angle between the axis X-X and axis O-O. Geometrically, angle α3 is equal to angle α2.

The device 20 has a first convex optical lens 32 located on the proximal end face 28 of the cylindrical optically transparent body 22. In the embodiment of FIG. 1, the first convex optical lens 32 is shown adhesively attached to the proximal end face 28 e.g., with a very thin layer of a special optical adhesive substance 29 (FIGS. 1A and 1B), which is known in the art. The device 20 is provided with a second convex optical lens 34, which is placed into a cavity 36 formed in the distal end face 30. Reference numeral 37 in FIG. 18 designate a layer of an adhesive substance that secures the convex lens 30 in the cavity 36 of the body 26. The side 38 of the lens 34 facing the gonioscopic eye support s coplanar with the plane P2 of the proximal end face 28 (FIG. 1A).

In the modification of the device 20 shown in FIGS. 1A and 1B, the gonioscopic lens eye support 24 has a flat rear side 40 and a tapered front side 42 with a cavity 42 which coincides in its shape with the cornea 27 of the eye 31 being investigated. During an ophthalmoscopy procedure, a cavity 43, which provided on the eye-contacting side of the gonioscopic lens eye support 24 and is capable of accommodating the cornea 27 of the eye 31, is gently brought into contact with eye cornea 27 through a layer of a cushioning agent 29 (FIG. 2). A drop of such a cushioning agent is applied onto the cornea prior to the procedure. The cushioning agent is used to protect the cornea of the eye from traumatization. During the procedure, the flat distal end face 30 of the gonioscopic lens 22 rests onto the flat rear side 40 of the gonioscopic lens eye support 24 so that the gonioscopic lens 22 and the gonioscopic lens eye support 24 form an integral optical device 20 (FIGS. 1A and 1B).

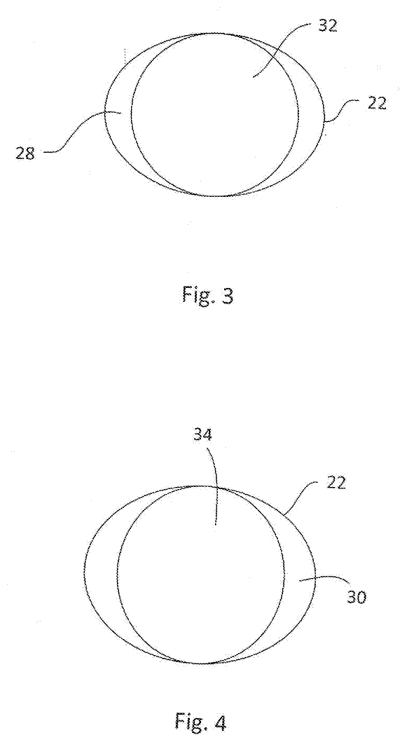

FIG. 3 is a view on the device 20 in the direction of arrow A of FIG. 2, and FIG. 4 a view on the device 20 in the direction of arrow B of FIG. 2. As mentioned above, reference numeral O-O designates the central symmetrical axis of the gonioscopic lens eye support 24 and during the procedure coincides with the optical axis of the eye (FIG. 2). Since the arrows A and are coaxial with the axis O-O, the end faces 28 and 30 will be seen as ovals, and the lenses 32 and 34 will be seen as circles, respectively, inscribed into the respective ovals.

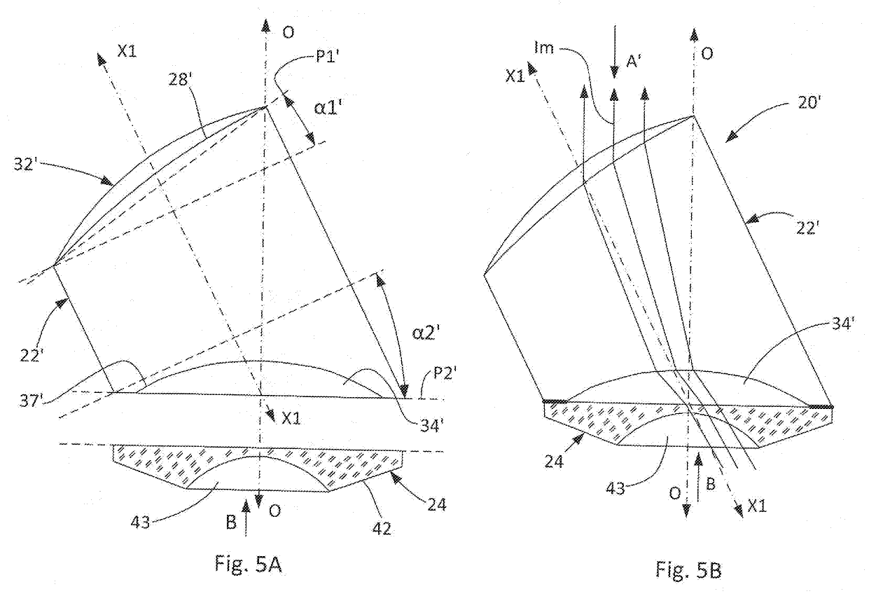

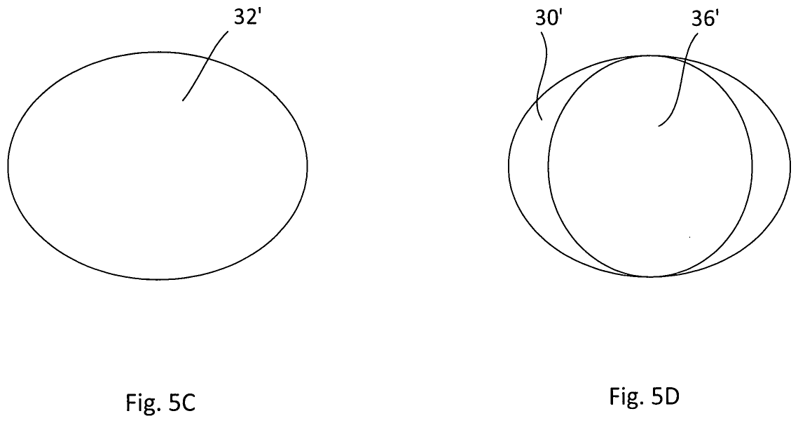

According to another modification, which is shown in FIG. 5A, the first convex optical lens 32′ can be made in a form that covers the entire surface of the proximal end face 28, However, the space of the gonioscopic lens body 26, which, in the embodiment of FIGS. 1A and 1B, was occupied by the lens 34, is empty and is formed as a cavity 36′. In fact, a curvilinear concave surface 37′ (FIGS. 5A and 5B) of the gonioscopic lens body 26 functions as a concave lens 34 and can be considered as such. The other elements of the gonioscopic device of FIG. 5A remain the same as in the modification of FIG. 1A and designated by the same reference numerals but with an addition of a prime (′). In FIG. 5B, the gonioscopic device 20′ is shown in an assembled state. FIG. 5C is a view in the direction of arrow A′, and FIG. 5D is a view in the direction of arrow B′.

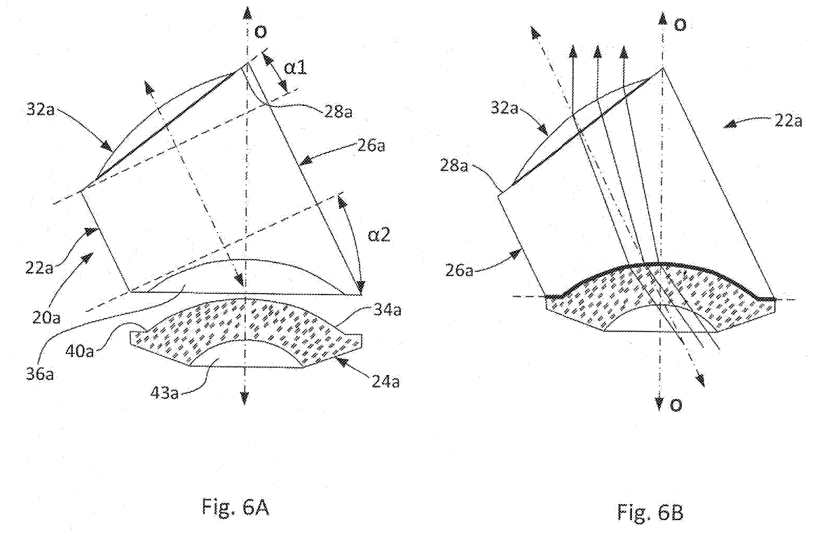

In the modification shown in FIG. 6A, the second convex lens 34a on the rear side 40a of the gonioscopic lens eye, support 24a, and in an assembled state (FIG. 6B) of the gonioscopic device 20a, the cavity 36a receives the lens 34a. The remaining parts of the gonioscopic device 20a of FIGS. 6A and 6B are the same as in the device of FIG. 1 and are designated by the same reference numerals with an addition of letter “a”.

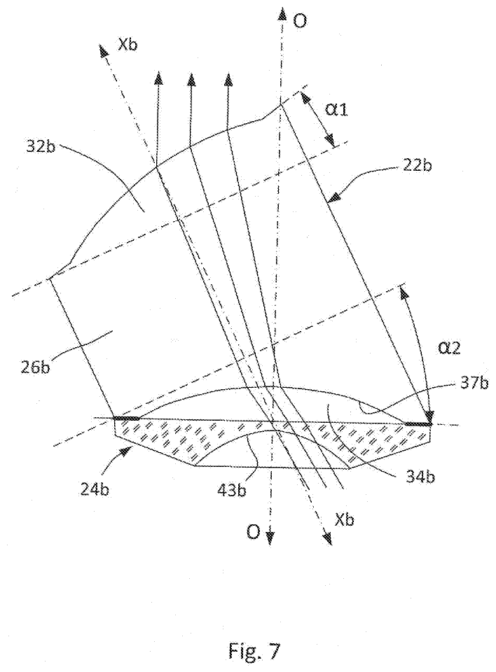

FIG. 7 shows a gonioscopic device 20b which is the same as one shown in FIG. 5A except that the first convex optical lens 32b is molded integrally with the gonioscopic lens 22b. All other elements of the gonioscopic device 20b are the same as in the modification of FIG. 5A and FIG. 5B and are, designated by the same reference numeral as FIGS. 5A and 5B but with an addition of letter “b”.

Let us consider now with reference to FIG. 2 some elements of tracing of rays passing through the optical system formed by the device of the invention, i.e., the gonioscopic lens 22 (22a, 22b, 22′) and the gonioscopic lens eye support 24 (24a, 24b, 24′). In ophthalmological examination, the main areas of interest are peripheral inner areas, i.e., anterior chambers and, particularly anterior chamber angles of the eye.

In FIG. 2, the areas of interest, i.e., anterior chamber angles of the eye 1, are designated by reference numerals 44 and 45. The ray tracing is shown with regard to the anterior chamber angles 44. Rays Im that exit the eye pass though the gonioscopic device 20 are optically conjugated with the pupil of the eye of the viewer V who sees the field of interest on his/her retina. It is understood that by changing geometric and material parameters of the device components it becomes possible to vary parameters of the image seen by the viewer. Such parameters are the following: dimensions of the gonioscopic lens, the gonioscopic lens eye support, curvatures of the lenses, refractor indices of the device components, etc. The changes of the aforementioned parameters define values of angles α1 and α2 wherein within the scope of the invention, angle α1 is in the range of 15° to 28°, preferably in the range of 18 to 22°, and angle α2 is in the range of 15° to 28°, preferably in the range of 18° to 22°. If the angles α1 and α2 are less than 15°, the anterior chamber angles 44 and 46 of the eye 31 will be beyond the range of observation (the ray tracing is shown only for the anterior chamber angle 44). If, on the other hand, the angles α1 and α2 exceed 28°, the rays Im will become angles, of complete internal reflection from the inner surface of the cornea, so that the viewer will see only the external surface of the cornea.

Regarding the optical materials of the gonioscopic device 20, the transparent body 26 (26a, 26b, 26′) of the gonioscopic lens can be made from various transparent glasses and plastics with refractive indices in the range of 1.45 to 2.00. The gonioscopic, lens eye support also can be made from various transparent glasses and plastics with refractive indices in the range of 1.45 to 2.00. The gonioscopic device 20 of the invention composed of two main elements, i.e., the gonioscopic lens and the gonioscopic lens eye support, opens great opportunities for varying optical properties of the device 20. This can be achieved by selecting various combinations of the lens geometric and material parameters of both components of the device. For example, the support 24 (24a, 24b, 24″) can be made disposable, e.g., if it is made from plastic such as polycarbonate. Alternatively, the support can be sterilizable if it is made, e.g., of glass, such as borosilicate glass Schott BK7.

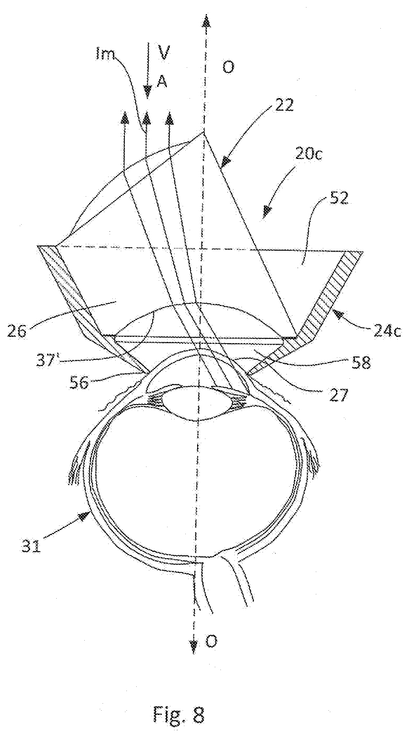

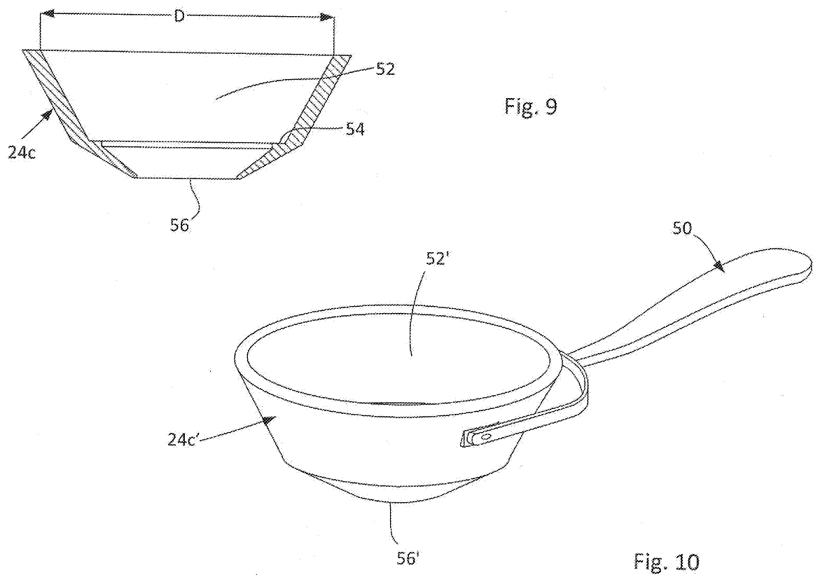

According to one or several other aspects of the invention, the gonioscopic lens eye support 24 may have a shape different from those shown in FIGS. 1 to 7 provided that it fulfills its function of supporting a gonioscopic lens 22. For example, as shown in FIGS. 8, 9, and 10 (where FIG. 8 is side view of a gonioscopic device 20c in a working position during the eye examination procedure, FIG. 9 is a transverse sectional view of a gonioscopic lens eye support 24c, and FIG. 10 is a three-dimensional gonioscopic lens eye support 24c, with a handle 50), the support 24c (24c) can be made in the form of a metal or non-transparent or transparent plastic funnel-shaped casing tapered in the downward direction, i.e., toward the eye 31 (FIG. 8). The support 24c (24c′) has a tapered cavity 52 (52′) with an inner circular step (or several intermittent steps) 54 (FIG. 9), which serves as a stopper for a gonioscopic lens 22 (FIG. 8), when the latter is inserted into the cavity 52 (52′). A diameter D of the support 24c (24c′) on the proximal end face 28c (FIG. 9) should exceed the diameter of the cylindrical body 26 to provide unobstructed insertion of the gonioscopic lens 22 into the cavity 52. A diameter of the support 24c (24c′) on its distal end face 56 (56′) is determined by the size of the eye cornea 27. This is because in the modification of FIGS. 8, 9, and 10, the lower circular end face 56 on the distal end of the device 20c (FIG. 8) rests, during an ophthalmic procedure (FIG. 8), on the peripheral area of the eye cornea 27. As a result, the entire area of the eye cornea, except a small peripheral area, is free of contact with the lens and thus is not subject traumatization. In other words, during a procedure, an air space 58 is formed over the eye cornea 27. If necessary, the contact area of the end face 56 with the eye may represent not a continuous ring, but several discretely located protrusions, Practice has shown that the presence of slots between the protrusions provides depressurization of the space above the eye cornea and prevents lens fogging.

Since during gonioscopic observation of an eye, an ophthalmologist need to rotate the device 20c on the eye 31, the support 24c′ may be provided with a handle 50 that facilitates manipulations with the device during the procedure.

Thus, in spite of differences in external appearance, the modifications of FIGS. 1 to 7 and 8 to 10 incorporate the same structural features and do not violate a unity of the invention. In other words, both devices are composed of a gonioscopic lens 22 (in a form of a transparent body of rotation (in this case, a cylindrical body) 26 having a longitudinal axis X-X, a proximal end face 28 on a viewer side tilted at an angle α1 to a plane that is normal to the longitudinal axis X-X of the cylindrical optically transparent body, and a distal end face 30 located on the side of an object to be observed and tilted to a plane that is normal to the longitudinal axis of the cylindrical optically transparent body at an angle α2. Both devices further contain a first convex optical lens 32 located on the proximal end face 30 of the cylindrical optically transparent body 26 and a second optical lens 34 (34a) or a curvilinear concave surface 37′ (FIGS. 5A, 58 and 8) of the gonioscopic lens body 26, which functions as a concave lens 4 and can be considered as such.

Furthermore, both devices 20 and 20c have a gonioscopic lens eye support 24 and 24c that supports the distal end face 30 of the optically transparent body 26 and has a cavity 43 is capable of accommodating the cornea 27 of the eye 31.

It should be noted that practically all existing gonio lenses work by reflecting the illuminating light from inclined flat mirrors of different designs. Therefore, during a procedure, it is necessary to use a slit lamp and, since a zone of illumination is about 2 square mm, it should be observed via a microscope. Contrary to this, in the case of the present invention, the area of the illuminated zone is large enough and is about 20 square mm. Therefore, a microscope is not needed.

The invention was described and illustrated with reference to specific drawings. It should be understood that the drawings are given only as examples and should not be construed as limiting the scope of application of the present invention. Therefore, any changes and modifications are possible provided that they do not depart from the scope of the attached patent claims. For example, the lenses can, be made from different transparent plastics or glass. The gonioscopic lens 22 may be a conical body of rotation. The gonioscopic lens, eye support may have configurations different from those shown in in the drawings. The device 20 shown in modifications of FIGS. 1 to 7 may be enclosed into a casing made from metal or plastic.

What I claim is:

1. An ophthalmic gonioscopic lens device with tilted lenses comprising:
a gonioscopic lens in a form of an optically transparent body of rotation having a longitudinal axis, a proximal end face on a viewer side tilted at an angle α1 to a plane that is normal to the longitudinal axis of the optically transparent body of rotation, and a distal end face located on the side of an object to be observed and tilted to a plane that is normal to, the longitudinal axis of the optically transparent body of rotation at an angle α2;
a first convex optical lens located on, the proximal end face of the optically transparent body of rotation;
a second optical lens located at the distal end face of the optically transparent body of rotation; and
a gonioscopic lens eye support that supports the distal end face of the optically transparent body of rotation and has a cavity capable of accommodating an eye to be examined and a coefficient of optical refraction close to that of the eye.

2. The ophthalmic gonioscopic lens device according to claim 1, wherein the optically transparent body of rotation is a cylindrical optically transparent body of rotation.

3. The ophthalmic gonioscopic lens device according to claim 2, wherein the first convex optical lens is made integrally with, the cylindrical optically transparent body.

4. The ophthalmic gonioscopic lens device according to claim 2, wherein cylindrical optically transparent body has a curvilinear concave cavity, which functions as the second optical lens.

5. The ophthalmic gonioscopic lens device according to claim 4, wherein the second optical lens is a concave lens and is made integrally with the cylindrical optically transparent body.

6. The ophthalmic gonioscopic lens device according to claim 2, wherein the second optical lens is a convex lens and is made integrally with the gonioscopic lens eye support on the side opposite to the recess.

7. The ophthalmic gonioscopic lens device according to claim 2, wherein the first convex optical lens is inscribed into the proximal end face of the cylindrical optically transparent body.

8. The ophthalmic gonioscopic lens device according to claim 2, wherein the second optical lens is inscribed into the distal end face of the cylindrical optically transparent body.

9. The ophthalmic gonioscopic lens device according to claim 2, wherein the angle α1 is in the range of 18° to 22°, and the angle α2 is in the range of 18° to 22°.

10. The ophthalmic gonioscopic lens device according to claim 3, wherein the angle α1 is in the range of 18° to 22°, and the angle α2 is in the range of 18° to 22°.

11. The ophthalmic gonioscopic lens device according to claim 4, wherein the angle α1 is in the range of 18° to 22°, and the angle α2 is in the range of 18° to 22°.

12. The ophthalmic gonioscopic lens device according to claim 2, wherein the cylindrical optically transparent body is made from, plastic or glass.

13. The ophthalmic gonioscopic lens device according to claim 12, wherein the cylindrical optically transparent body is made from borosilicate glass Schott BK7.

14. The ophthalmic gonioscopic lens device according to claim 2, wherein the gonioscopic lens eye support is made from a sterilizable optical material.

15. The ophthalmic gonioscopic lens device according to claim 11, wherein the gonioscopic lens eye support is made from a sterilizable optical material.

16. The ophthalmic gonioscopic lens device according to claim 2, wherein the gonioscopic lens eye support comprises a funnel-shaped casing tapered toward an eye to be examined, the gonioscopic lens eye support having an inner surface and an inner circular step on the inner surface for supporting the gonioscopic lens when the latter is inserted the funnel-shaped casing.

17. The ophthalmic gonioscopic lens device according to claim 4, wherein the gonioscopic lens eye support comprises a funnel-shaped casing tapered toward an eye to be examined, the gonioscopic lens eye support having an inner surface and an inner circular step on the inner surface for supporting the gonioscopic lens when the latter is inserted into the funnel-shaped casing.

18. The ophthalmic gonioscopic lens device according to claim 17, wherein the funnel-shaped casing further comprising a handle that facilitates manipulations with the device.

19. The ophthalmic gonioscopic lens device according to claim 1 wherein the gonioscopic lens eye support comprises a funnel-shaped casing tapered toward an eye to be examined, the gonioscopic lens eye support having an inner surface and an inner circular step on the inner surface for supporting the gonioscopic lens when the latter is inserted into the funnel-shaped casing.

20. The ophthalmic gonioscopic lens device according to claim 19, wherein the funnel-shaped casing further comprising a handle that facilitates manipulations with the device.
